(19) United States
(12) Patent Application Publication
(10) Pub. No.: US 2010/0216915 A1
(43) Pub. Date: Aug. 26, 2010

(54) Levulinic acid ester derivatives as reactive plasticizers and coalescent solvents

(75) Inventor: Paul D. Bloom, Decatur, IL (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Archer-Daniels-Midland Company, Decatur, IL (US)

(21) Appl. No.: 11/655,894

(22) Filed: Jan. 22, 2007

Related U.S. Application Data
(60) Provisional application No. 60/760,371, filed on Jan. 20, 2006.

(51) Int. Cl. C08L 3/02; C08L 5/00; C08K 5/053; C08K 5/1535; C08K 5/1545; C08K 5/07; C07D 307/02; C07C 43/03; C07C 31/18; C07D 309/02; C07D 493/04; C07C 69/66; C12P 17/18; C12P 17/06; C12P 17/04; C12P 7/18; C08L 1/00

(52) U.S. Cl. 524/35; 524/27; 524/386; 524/111; 524/377; 524/110; 524/357; 549/502; 568/680; 568/852; 549/417; 549/464; 560/176; 435/119; 435/125; 435/126; 435/158; 524/47

(57) ABSTRACT

The present invention is directed to ester derivatives of levulinic acid that are useful as plasticizers and/or coalescent solvents in polymer compositions, compositions comprising the ester derivatives, methods of making the derivatives and the compositions, and the use of the derivatives as additives in polymer compositions.

### CROSS REFERENCE TO RELATED APPLICATIONS

This application claims the benefit of U.S. Provisional Application No. 60/760,371, filed Jan. 20, 2006, which is incorporated by reference herein in its entirety.

### STATEMENT REGARDING FEDERALLY-SPONSORED RESEARCH AND DEVELOPMENT

Part of the work performed during development of this invention utilized U.S. Government funds. The U.S. Government has certain rights in this invention under grant number DE-FC36-04G014158 awarded by the Department of Energy.

### BACKGROUND OF THE INVENTION

1. Field of the Invention

Ester derivatives of levulinic acid as described herein are useful as reactive plasticizers and coalescent solvents, and can reduce the amount of volatile organic compounds (VOCs) when used to replace traditional plasticizers and coalescent solvents in polymer compositions, plastics and water-based coatings.

2. Background of the Invention

Coating compositions, such as paints, that include latex polymer particles, such as resins, typically also include a coalescent (i.e., coalescing agent, cosolvent or film-forming agent) in addition to pigments and fillers. The coalescent functions as a solvent as well as a plasticizer for the polymer particles to soften the latex polymer particles and assist in the formation of a continuous coating or film after applying to a surface and allowing to dry.

Useful coalescents are generally stable in the presence of water, compatible with other ingredients typically used in paint formulations, particularly the latex polymers, such that the stability of the latex-based composition is not compromised. They are also typically sufficiently volatile to escape when the applied coating composition is allowed to dry, but sufficiently nonvolatile to evaporate more slowly than other ingredients (e.g., drying retarders, antifreezes) that delay film formation. However, there is a general desire in the industry to reduce volatile organic emissions, thereby reducing the environmental and health concerns.

Governments have established regulations setting forth guidelines relating to volatile organic compounds that may be released into the atmosphere. To reduce the level of volatile organic compounds, high solids coating compositions have been developed as well as powder coating compositions that do not include volatile compounds, have a reduced concentration of volatile compounds, or incorporate compounds having a lower volatility. Reducing the volatility of coalescents, solvents, plasticizers, etc. can adversely affect the balance of properties needed in a latex-based coating composition, however. Thus, there is a need for coalescents, solvents, plasticizers, etc. that can be used in coating compositions, such as paints, that do not compromise stability, compatibility, film formation ability, or the desirable properties of the applied coating.

Aqueous dispersions of particulate polymer, such as a resin, or emulsified liquid pre-polymers for use as paints, sealants, caulks, adhesives or other coatings are well-known, widely-used articles of commerce. The effectiveness of the dispersion in forming a film after the polymer dispersion has been deposited upon a surface depends upon the glass transition temperature (T_{g}) of the dispersed polymer and the temperature at which the film is allowed to dry. See, e.g., Conn et al., U.S. Pat. No. 2,795,564, and Emmons et al., U.S. Pat. No. 4,131,580.

Coalescent aids have been used in such aqueous dispersions to soften, i.e., plasticize, the particulate polymers and facilitate the formation of a continuous film with optimum film properties once the water has evaporated. In addition to increasing the ease of film formation, the coalescent aid also promotes subsequent improvements in film properties by coalescing the particulate polymers and liquid pre-polymers and forming an integral film at ambient temperatures. Without the coalescent aid, the films may crack and fail to adhere to the substrate surface when dry.

Coalescent aids are particularly helpful in assisting the formation of particulate polymer films possessing a high glass transition temperature, that is, the temperature which defines how easily the particles of the polymer diffuse at the temperature at which the film-forming composition is applied. The presence of coalescent aids in a particulate polymer film having a high glass transition temperature allows optimum film formation at ambient temperatures.

Various alcohol esters and ether alcohols have been proposed for use as coalescent aids. For example, in U.S. Pat. No. 4,131,580, Emmons et al. disclose water-based coating compositions based on vinyl addition polymers of monoethylenically unsaturated monomers which comprise dicyclopentenyl acrylate and/or dicyclopentenyl methacrylate as a coalescent aid. In U.S. Pat. No. 4,141,868, Emmons et al. suggest certain ester-ether compounds be used instead. Two of the more widely used coalescent aids are ethylene glycol monobutyl ether (EB, Union Carbide) and 2,2,4-trimethyl-1,3 pentanediol monobutyrate (TEXANOL, Eastman Kodak). Both EB and TEXANOL are useful in facilitating film formation of particulate polymer coatings with high glass transition temperatures and are even useful in facilitating film formation of particulate polymer coatings with low glass transition temperatures if they are being applied at a temperature that is lower than ambient temperature. However, they are relatively volatile and, as a result, are currently classified as VOCs (volatile organic compounds).

Both legislative and marketplace developments are pushing for reduced volatile organic emissions in a variety of industries. In an increasing number of industries, aqueous coating compositions continue to replace solvent-based coating compositions in efforts to significantly reduce volatile organic emissions. A variety of paints, inks, sealants and adhesives, for example, which were previously formulated with organic solvents are now formulated as aqueous compositions. Emissions from coatings compositions commonly result from volatile organic compounds (VOCs) in the compositions. The amounts of VOCs in a coating composition are expressed in grams per liter (g/L).

While the move from organic solvent-based to aqueous compositions brings environmental, safety and health benefits, aqueous coating compositions must still meet or exceed the performance standards expected from solvent-based coating compositions. The coatings or films must form at ambient temperatures (35° to 160° F.), yet have good performance properties after curing. For example, a coating composition should exhibit good print and block resistance and yield good adhesion and tensile properties. Once cured, most applications require that the coating be unaffected by environmental conditions such as water, humidity, and end-use temperature fluctuations.

Aqueous coating compositions may contain upwards of ten to twenty components which are generally identified by their function. For example, in addition to a resin or resins (also called latexes or binders, including but not limited to acrylic emulsion latex polymer), an aqueous coating composition may have pigments, extenders, antisettling agents, dispersants, surfactants (such as wetting agents, defoamers, and antifoamer), rheology modifiers, coalescing solvents, plasticizers, water, glycols, catalysts, biocides, crosslinkers, and colorants. Resins have a typical solid content, referred to as “resin solids.” Glycols are components added for freeze-thaw resistance, wet edge properties and as aids in low temperature coalescence. Representative glycols used for these purposes include ethylene glycol and propylene glycol. Because the glycols generally evaporate at ambient conditions, they contribute to VOCs found in aqueous coating formulations. A typical contribution to VOCs by glycols would be 100 to 200 grams per liter. Glycols are one of the first components aqueous coating manufacturers seek to decrease or eliminate in an effort to reduce emissions. However, the resulting coating may then suffer in the desired properties of low temperature coalescence, freeze/thaw resistance and wet edge.

Cosolvents (also known as coalescing solvents) are commonly employed in aqueous compositions to aid in film formation (or knitting-together) of hard latex particles. This hardness can be measured in terms of the starting film-formation temperature or of the glass transition temperature of the manufactured latex solid. As drying occurs, the cosolvents evaporate from the coating and the glass transition temperature of the coating approaches that of the starting resin. The addition of cosolvents enables the coating to behave like a softer film-forming material during drying and then perform as a harder, resistant film after drying. Examples of cosolvents include aliphatic and aromatic hydrocarbons and oxygenated solvents, such as alcohols, ketones and glycol ethers. A typical amount of cosolvent ranges from 50 to 300 or more grams per liter of coating composition.

Because cosolvents that are present in the coating formulation contribute considerably to VOC content and tend to contribute odor to the coating, cosolvents are becoming more undesirable in aqueous coatings. However, in many aqueous coating systems, the elimination of the cosolvent(s) result in either lack of film formation or such poor film formation that the coating has poor appearance and poor resistance properties. In some cases, plasticizers may be added to the aqueous coating formulation to replace some or all of the cosolvents. Typically, plasticizers are organic compounds that do not significantly evaporate at ambient conditions but remain in the coating. Examples of typical plasticizers would be chemicals from the phthalate, adipate, and benzoate families. They soften the polymer and are used to impart flexibility to an otherwise hard and brittle polymer. However, plasticizers, especially at high levels, can have deleterious effects on coating performance properties. Because the coating remains soft, it can have poor block and print resistance, poor stain resistance and a tacky feel.

Many of the components discussed above used to formulate waterborne coatings have small amounts of volatile compounds present along with the components. Some examples are the solvents that colorants are dispersed in, the solvents that catalysts are dispersed in, and even the glycols or solvents that are present in many commercial surfactants, biocides, defoamers, or rheology modifiers.

As discussed above, a need exists to reduce or eliminate VOCs from aqueous coating compositions without adversely affecting properties such as wet coating, T_{g} and end-use performance.

### SUMMARY OF THE INVENTION

The present invention is directed to ester derivatives of levulinic acid that are useful as plasticizers and/or coalescent solvents in polymer compositions, compositions comprising the ester derivatives, methods of making the derivatives and the compositions, and the use of the derivatives as additives in polymer compositions.

### DETAILED DESCRIPTION OF THE INVENTION

The present invention is directed to ester derivatives of levulinic acid that are useful as plasticizers and/or coalescent solvents in polymer compositions, compositions comprising the ester derivatives, methods of making the derivatives and the compositions, and the use of the derivatives as additives in polymer compositions.

In an embodiment, the invention is directed to a composition comprising a) a polymer; and b) a coalescent solvent and/or plasticizer comprising one or more compounds selected from the group consisting of a polysaccharide, a polyol, a residue of a polysaccharide or a polyol, or a mixture thereof directly covalently linked by an O or N with one or more of -(A) or -(G); wherein said -(A) and said -(G) have the following formulae:

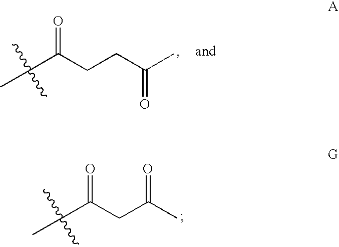

The term “polysaccharide” refers to a carbohydrate that can be decomposed by hydrolysis into two or more molecules of monosaccharides, especially any of the more complex carbohydrates (as cellulose, starch, fibers, or glycogen).

The term “polyol” refers to a polyhydric alcohol, i.e., one containing two or more hydroxyl groups. A polyol includes compounds with the general formula CH₂OH(CHOH)ₙCH₂OH, where n may be from 0 to 5. Illustrative polyols also include sugar alcohols, such as sorbitol, mannitol, erthritol, lactitol, maltitol and xylitol, glycerols, glycols, dialkylethers substituted with at least two hydroxyl groups, heterocylic alcohols, such as furan alcohols, pyran alcohols, tetrahydrofuran alcohols, tetrahydropyran alcohols, oxacyclohexane alcohols, dianhydrohexitols, and aldehyde alcohols, such as hexanal and pentanal alcohols. Further examples of polyols include compounds B, B′, C, D, E, F and H, described below.

The term “residue of a polysaccharide or a polyol” refers to polysaccharides, polyols, and other related compounds derived from polysaccharides or polyols. Residues of polysaccharides or polyols include polysaccharides and polyols, including those listed above, monosaccharides (i.e., a sugar not decomposable to simpler sugars by hydrolysis), monohydric alcohols, mixtures and derivatives thereof. Starches and cellulosic compounds can be hydrolyzed or can undergo catalytic cracking (also called hydrogenolysis) to provide residues of polysaccharides and polyols. Residues of polysaccharides or polyols can be derived from a variety of sources, as discussed below.

Examples of such residues including polysaccharides and monosaccharides include, but are not limited to, arabinose, xylose, sucrose, maltose, isomaltose, fructose, mannose, lactose, galactose, glucose, glucosides, for example alpha-d-alkyl glucosides such as alpha-methyl-d-glucoside, pentaacetylglucose and gluconic lactone.

Other examples of residues a polysaccharide or a polyol include furans, tetrahydrofurans, dianhydrohexitols, pyrans, tetrahydropyrans and oxacyclohexanes substituted by one or more ether, hydroxy, amine, ester, aldehyde or keto groups. Further examples include dialkyl ethers substituted by one or more ether, ester or hydroxy groups, alkanes substituted by one or more ether, hydroxy groups or ester groups, hexanals or pentanals substituted by one or more hydroxy, ether or ester groups and oxacyclohexanes substituted by one or more hydroxy or ether groups. Specifically, residues of polysaccharides or polyols include compounds I-XIX, discussed below, without attachment to the levulinic compound.

Other residues of polysaccharides and polyols comprise molecules containing hydroxyl groups and can include high fructose corn syrup, and sugars obtained therefrom.

The coalescent solvent and/or plasticizer of (b) can include one or more compounds having the following structures:

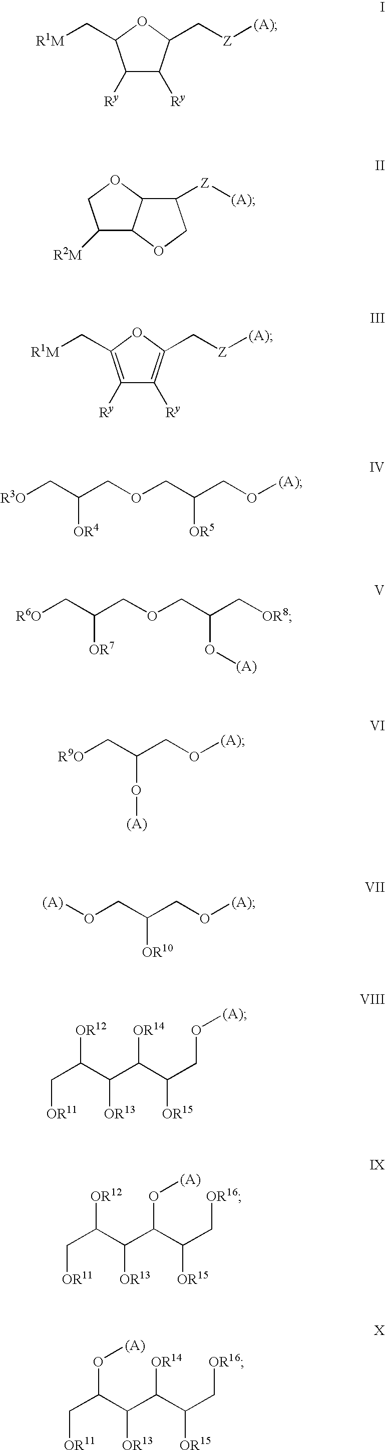

wherein, R¹, R², R³, R⁴, R⁵, R⁶, R⁷, R⁸, R⁹, R¹⁰, R¹¹, R¹², R¹³, R¹⁴, R¹⁵ and R¹⁶ are each independently selected from the group consisting of hydrogen, (A)-, C₁₋₄ alkyl, benzyl, benzoyl and

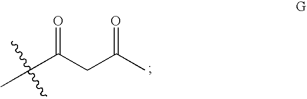

### M=O or N; and

### Z=O or N,

wherein values of M and Z are independently selected, and M and Z can be the same or different;

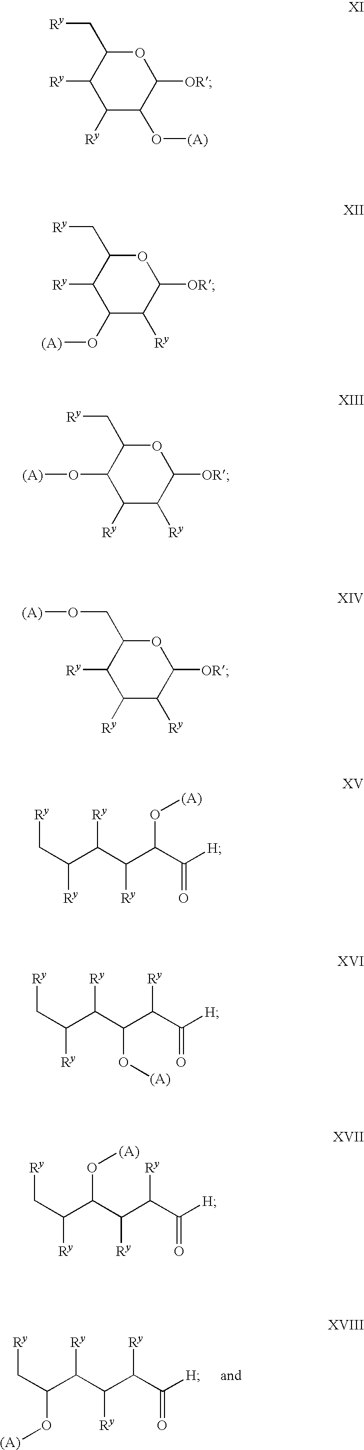

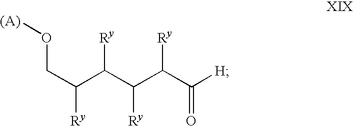

wherein R^{y} in each instance is independently selected from the group consisting of hydroxy, hydrogen, (A)-O—, (G)-O—, C₁₋₄ alkoxy, benzyloxy and benzoyloxy; R′ is independently selected from the group consisting of hydrogen, (A)-, C₁₋₄ alkyl, benzyl, -(G), and benzoyl; G, in each instance, is:

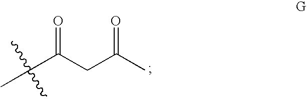

and A, in each instance, is:

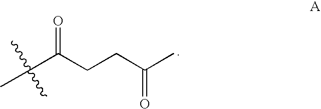

In an embodiment, the coalescent solvent and/or plasticizer can be the structure of Formula XI, wherein each R^{y} group is hydroxyl and R′ is C₁₋₄ alkyl.

Each of the above structures (i.e., polysaccharide, polyol or a residue of a polysaccharide or polyol) contains a levulinic acid residue (A). In all instances described herein, A is as shown above. The levulinic acid residue of the molecule provides a reactive site for hydrazide (dihydrazide or polyfunctional hydrazides) crosslinking of the coalescent or plasticizer to the polymer. See, e.g Water-curable resin compositions for coating materials, Aoki, et al. JP05148406A2, 19930615 Heisei, Application: JP91-337971. Such crosslinking can provide increased durability, hardness and/or solvent resistance to the dried coating film. The above structures also possess properties of coalescent solvents and/or plasticizers for polymer compositions.

The use of the terms “coalescent solvent” and “plasticizer” refer to the property of the compounds, and compositions comprising the compounds. These compounds and compositions are capable of softening the polymer, lowering the glass transition temperature, T_{g}, of the composition and/or promoting formation of a continuous film. The levulinic acid ester derivatives are non-volatile and when used to replace traditional coalescent solvents and plasticizers, can reduce the amount of VOCs needed in the polymer composition.

In another embodiment, levulinic acid ester derivatives can be prepared from starches and cellulosic compounds. The hydroxy groups on starches and cellulosic compounds can be esterified with levulinic acid or transesterified with a levulinate ester. The starch or cellulosic compounds can be further functionalized with levulinic acid and/or fatty acids. A polyunsaturated fatty acid can be incorporated in the levulinic acid ester derivative of this type to provide oxidative properties, such as oxidative cross-linking. Starch functionalized with levulinic acid or fatty acids can provide more than one desirable property in film forming compositions, such as modification of rheological properties, such as thickening, combined with reactive coalescent properties.

In another embodiment, mixed polyols can be esterified with levulinic acid or transesterified with a levulinate ester. Natural fibers can by hydrolyzed to provide residues of polysaccharides and polyols, such as mixtures of polyols. Fibers suitable for this purpose include, but are not limited to, corn fiber from corn wet mills, dry corn gluten feed which contains corn fiber from wet mills, wet corn gluten feed from wet corn mills that do not run dryers, distiller dry grains solubles (DDGS) and Distiller's Grain Solubles (DGS) from dry corn mills, canola hulls, rapeseed hulls, peanut shells, soybean hulls, cottonseed hulls, cocoa hulls, barley hulls, oat hulls, wheat straw, corn stover, rice hulls, starch streams from wheat processing, fiber streams from corn mesa plants, edible bean molasses and edible bean fiber. Hydrolyzates of natural fibers, such as corn fiber, are enriched in residues of polysaccharides and polyols including but not limited to arabinose, xylose, sucrose, maltose, isomaltose, fructose, mannose, galactose, and glucose. The residues of polysaccharides and polyols obtained by hydrolysis of polyols can be esterified with levulinic acid or transesterified with a levulinate ester. The residues of polysaccharides and polyols obtained by hydrolysis of polyols can be further functionalized with levulinic acid/fatty acids. A polyunsaturated fatty acid can be incorporated in the levulinic acid ester derivative of this type to provide oxidative properties, such as oxidative cross-linking.

Residues of polysaccharides and polyols obtained from hydrolyzed fibers may be subjected to fermentation. The fermentation process may provide new polyols, or may alter the amounts of residues of polysaccharides or polyols obtained from hydrolyzed fibers. After fermentation, a fermentation broth is obtained, and residues of polysaccharides or polyols can be recovered and/or concentrated from the fermentation broth to provide a natural feedstock of biological origin suitable for esterification with levulinic acid or polyunsaturated fatty acids, or transesterification with a levulinate ester. A polyunsaturated fatty acid can be incorporated in the levulinic acid ester derivative of this type to provide oxidative properties, such as oxidative cross-linking.

In another embodiment, residues of polysaccharides or polyols obtained from catalytic cracking of mixed polysaccharides or polyols can be esterified with levulinic acid or transesterified with a levulinate ester. In an embodiment, residues of polysaccharides and polyols obtained by catalytic cracking of a mixed polysaccharide or polyol source comprising corn fiber hydrolyzate can be esterified with levulinic acid or levilunate ester. Polysaccharides and polyols, such as hydrolyzates of natural fibers, such as corn fiber, are enriched in residues of polysaccharides and polyols including but not limited to arabinose, xylose, sucrose, maltose, isomaltose, fructose, mannose, galactose, and glucose. After catalytic cracking the reaction products are enriched in residues of polysaccharides and polyols having 2-5 carbons. The residues of polysaccharides and polyols obtained by catalytic cracking of polyols can be esterified with levulinic acid or transesterified with a levulinate ester. The residues of polysaccharides and polyols obtained by catalytic cracking of polyols can be further functionalized with levulinic acid/fatty acids. A polyunsaturated fatty acid can be incorporated in the levulinic acid ester derivative of this type to provide oxidative properties, such as oxidative cross-linking.

In another embodiment, residues of polysaccharides and polyols obtained by subjecting sugars or carbohydrates to catalytic cracking (also called hydrogenolysis) can be esterified with levulinic acid or levilunate ester. In a non-limiting example, sorbitol is subjected to catalytic cracking to provide a mixture of polyols, including polyols having 2-5 carbons (see, e.g. Hydrogenolysis of sorbitol. Clark, I. J. Ind. Eng. Chem. (Washington, D.C.) (1958), 50 1125-6). Other polysaccharides and polyols suitable for catalytic cracking include, but are not limited to, glucose, sorbitol, mannitol, sucrose, lactose, maltose, alpha-methyl-d-glucoside, pentaacetylglucose and gluconic lactone (see, e.g. Hydrogenolysis of sugars. Zartman, W. and Adkins, H. J. Amer. Chem. Soc. (1933) 55, 4559-63). The residues of polysaccharides and polyols obtained by catalytic cracking of polysaccharides and polyols can be esterified with levulinic acid or levulinate ester. The residues of polysaccharides and polyols obtained by catalytic cracking of polyols can be further functionalized with levulinic acid/fatty acids. A polyunsaturated fatty acid can be incorporated in the levulinic acid ester derivative of this type to provide oxidative properties, such as oxidative cross-linking.

The levulinic acid ester derivative is present in the polymer composition at an amount from about 1% to about 25% w/w total solids, for example, from about 5% to about 20% w/w total solids, or from about 7% to about 15% w/w total solids.

The T_{g} of the polymer composition is from about 1° C. to about 40° C., for example, from about 3° C. to about 20° C., or from about 5° C. to about 15° C.

The present invention is also directed to a method of lowering the T_{g} of a polymer composition by adding an effective amount of a levulinic acid ester derivative to the composition. The T_{g} of the composition comprising a levulinic acid will be lower than the T_{g} of the composition as measured prior to adding the derivative. An “effective amount” is any amount capable of lowering the T_{g} by not less than about 2° C. In an embodiment, the method of lowering the T_{g} of a polymer composition comprises adding a levulinic acid ester derivative such that the T_{g} is lowered by not less than about 5° C., for example, by not less than about 10° C.

The present invention can be used with any suitable polymer. Suitable polymers include polyhydroxyalkanoate, polyvinylchloride, acrylic polymers, styrene-acrylic, vinyl-acrylic, ethylene-vinyl acetate, styrene, polyesters, polyamides, polyethers, polybutadienes, Acrylonitrile-Butadiene-Styrene (ABS), Styrene-Butadiene-Styrene (SBS), and latex polymers, including acrylic latex emulsion polymers.

In another aspect, the present invention is directed to a method of preparing a polymer composition containing a levulinic acid ester derivative, the method comprising, combining a polymer as described above with a levulinic acid ester derivative, wherein a polymer composition is prepared.

In another aspect, the present invention is directed to compositions comprising one or more compounds of Formulae I, II, III, IV, V, VI, VII, VIII, IX, X, XI, XII, XIII, XIV, XV, XVI, XVII, XVIII, XIX, XX, XXI, XXII and XXIII as shown above.

In yet another aspect, the present invention is directed to compounds having the following Formulae I, II, III, IV, V, VIII, IX, X, XI, XII, XIII, XIV, XV, XVI, XVII, XVIII and XIX:

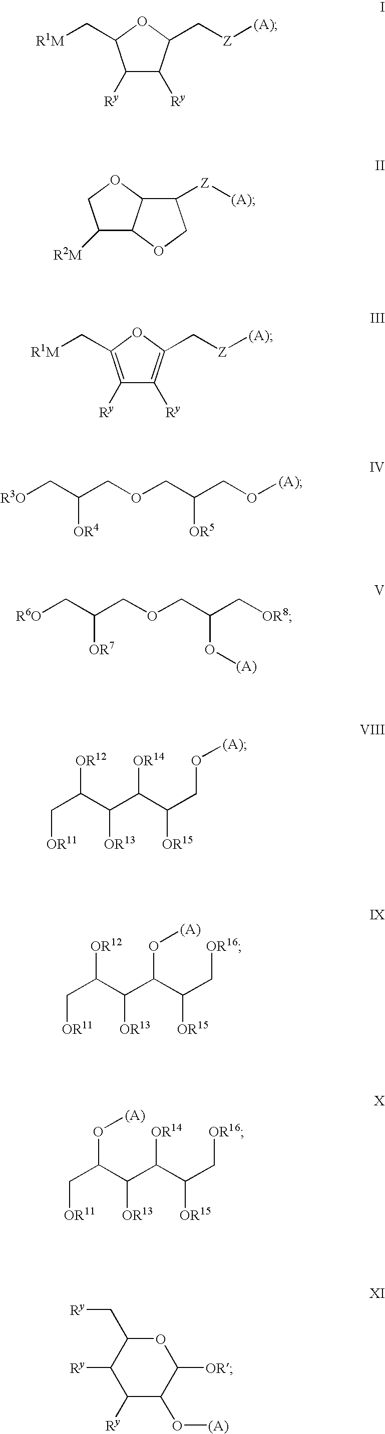

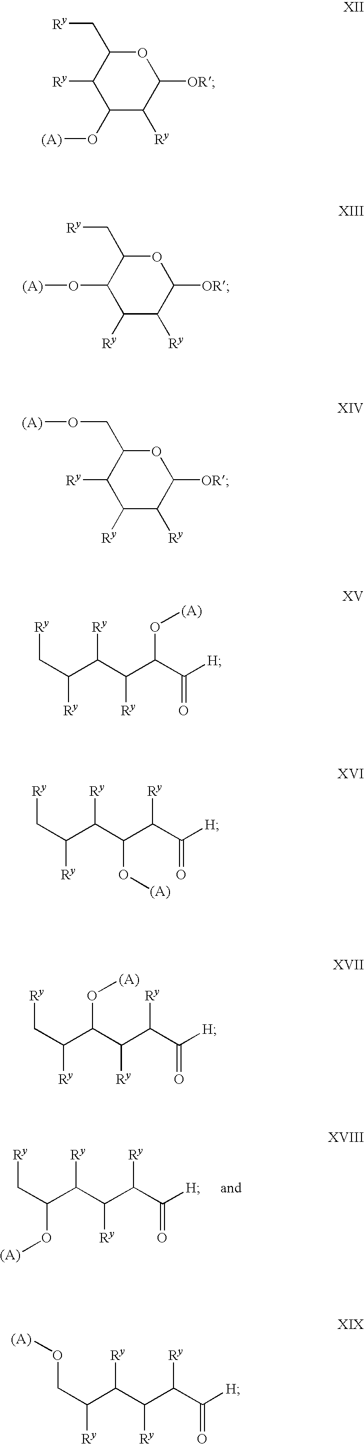

wherein, R^{y} in each instance is independently selected from the group consisting of hydroxy, hydrogen, (A)-O—, (G)-O—, C₁₋₄ alkoxy, benzyloxy and benzoyloxy; R′ is independently selected from the group consisting of hydrogen, (A)-, C₁₋₄ alkyl, benzyl and benzoyl; R¹, R², R³, R⁴, R⁵, R⁶, R⁷, R⁸, R¹¹, R¹², R¹³, R¹⁴, R¹⁵ and R¹⁶ are each independently selected from the group consisting of hydrogen, (A)-, C₁₋₄ alkyl, benzyl, benzoyl and

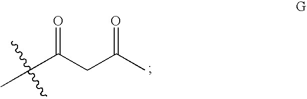

and A, in each instance, is:

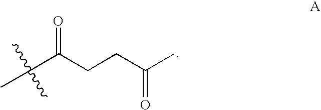

Especially useful compounds of Formula I include:

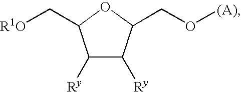

wherein, R¹ is selected from the group consisting of: hydrogen, C₁₋₄ alkyl, (G)-, and (A)-; and R^{y} is selected from the group consisting of: hydrogen and hydroxy.

A more preferred compound of Formula I has the following structure, Formula XX:

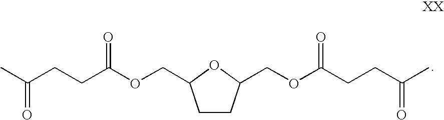

Especially useful compounds of Formula II include:

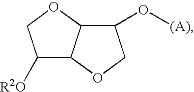

wherein, R² is selected from the group consisting of: hydrogen, C₁₋₄ alkyl, (G)- and (A)-. A more preferred compound of Formula II has the following structure, Formula XXI:

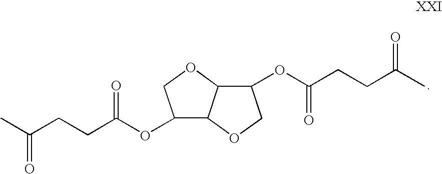

Especially useful compounds of Formula IV include:

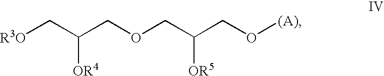

wherein, R³, R⁴ and R⁵ are each independently selected from the group consisting of hydrogen and (A)-. A more preferred compound of Formula III has the following structure, Formula XXII:

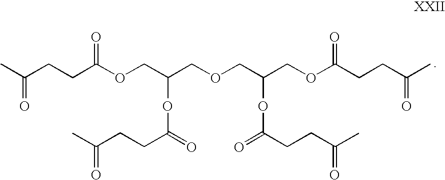

Especially useful compounds of Formula VIII include:

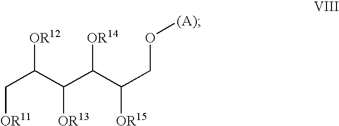

wherein, R¹¹, R¹², R¹³, R¹⁴ and R¹⁵ are each independently selected from the group consisting of hydrogen and (A)-. A more preferred compound of Formula VIII has the following structure, Formula XXIII:

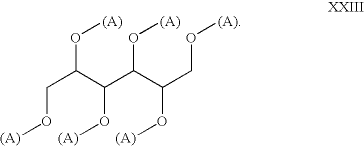

In another aspect, the present invention is directed to a method of making an ester derivative of levulinic acid comprising: a) in the presence of an acid catalyst, forming a mixture by combining levulinic acid and one or more of the following compounds:

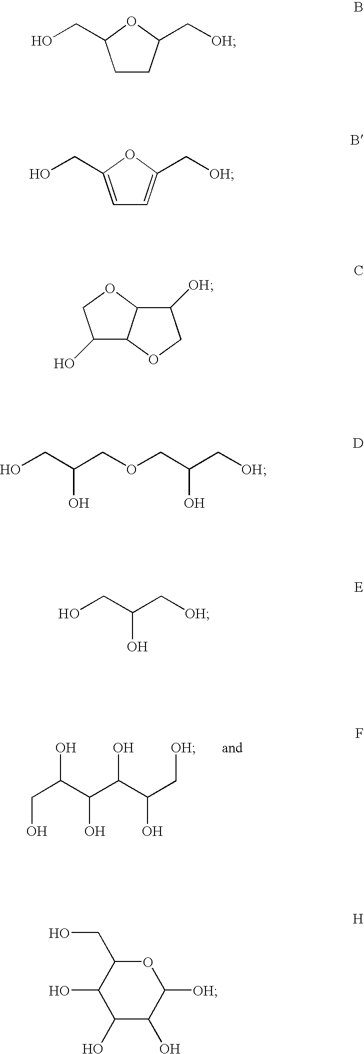

and b) heating said mixture at a temperature from about 50° C. to about 200° C. wherein a levulinic acid ester derivative is prepared. Useful temperatures also include temperatures from about 100° C. to about 150° C., for example, from about 110° C. to about 140° C. The acid catalyst employed can be sulfuric acid, p-toluenesulfonic acid, methanesulfonic acid, BF₃, acidic resin catalysts, zeolites, or organotin catalysts. In an embodiment, the catalyst is an acid that is a solid material.

Alternatively, the present invention is directed to a method of making an ester derivative of levulinic acid comprising: a) in the presence of a base catalyst, forming a mixture by combining levulinic acid ester and one or more of compounds B, B′, C, D, E, F, and H. Alternatively, the present invention is directed to a method of making an ester derivative of levulinic acid comprising: a) in the presence of an enzyme catalyst, forming a mixture by combining levulinic acid or a levulinic acid ester and one or more of compounds B, B′, C, D, E, F, and H.

A suitable enzyme would include a lipase, such as a triacylglycerol acylhydrolase (EC 3.1.1.3). A suitable lipase would include Novozym 435, available from Novozymes, Bagsvaerd, Denmark. When a lipase catalyst is used, moderate reaction temperatures (25-90° C.) can be employed.

Following the heating step, the mixture can be cooled or allowed to cool to about room temperature. The mixture can optionally be contacted with MgSO₄ and/or activated carbon, or similar compounds known in the art. The mixture can also be washed with water to remove byproducts from the organic phase, which will contain the ester derivative. The organic phase can be contacted with MgSO₄ or similar compounds known in the art, and separated. Purification of the mixture can include one or more for the following steps: liquid-liquid extraction, evaporation of excess levulinic acid or levulinate ester using wiped-film evaporators, molecular distillation and chromatographic methods. In another reaction scheme, anhydrous ethyl levulinate can be transesterified with an anhydrous alcohol or polyol under basic conditions using standard catalysts including sodium hydroxide, sodium methoxide or solid base catalysts including strong base resins.

### Schemes

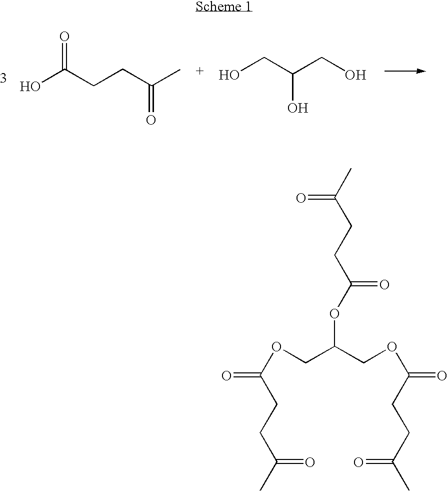

Scheme 1 depicts a method of preparing a trilevulinate of glycerol (a levulinic acid triester derivative). Glycerol trilevulinate (1,2,3-propanetriyl ester, 4-oxo-penanoic acid) was prepared using a solid acid catalyst under dehydration conditions. The solid catalyst was filtered and the reaction mixture was separated in water/ethyl acetate. The product was extracted into the ethyl acetate layer. The aqueous layer contained levulinic acid and partially esterified glycerol esters.

### EXAMPLES

### Example 1

### Synthesis of Glycerol trilevulinate (1,2,3-propanetriyl ester, 4-oxo-penanoic acid)

About 9.89 g glycerol (0.11 mol) (Aldrich), 102.6 g levulinic acid (0.88 mol) and 4 g Amberlyst 35 (Rohm and Haas) were added to a 3-neck round bottom flask equipped with a Barret style receiver, thermocouple and N₂ purge. The mixture was heated at about 128° C. with stirring for about 2.5 hours. The yellow liquid mixture was cooled to room temperature, dried over anhydrous MgSO₄ and contacted with activated carbon. The mixture was then washed with water. The organic layer was dried over anhydrous MgSO₄ and filtered to yield a clear liquid. The product had an acid value of 0.6 and was identified by NMR analysis as Glycerol trilevulinate (1,2,3-propanetriyl ester, 4-oxo-penanoic acid).

### Example 2

### Formulation of Polymer Composition Comprising Glycerol tri-levulinate (1,2,3-propanetriyl ester, 4-oxo-penanoic acid)

SG-10M (Rohm and Haas) polymer 50% resin solids and glycerol trilevulinate (1,2,3-propanetriyl ester, 4-oxo-penanoic acid) was mixed at levels described in Table 1 below.

TABLE 1

| | Resin Solids | Plasticizer | % Plasticizer on |
|---|---|---|---|
| Sample | (g) | (g) | total solids (w/w) |
| 1 | 10 | 0 | 0 |
| 2 | 10 | 1 | 9.1 |
| 3 | 10 | 2 | 16.7 |
| 4 | 10 | 3 | 23.1 |

After mixing, the samples were allowed to equilibrate overnight. Destabilization of the latex particles occurred in Sample 4 causing the sample to gel after 16 hours. Samples 1, 2 and 3 were cast into films on release paper using a 3 mil drawdown bar. The films were allowed to cure at room temperature for 96 hours. All films were transparent. Sample 3 showed presence of some exuded plasticizer on the surface of the film.

### Example 3

### Analysis of Films by Differential Scanning Calorimetry

The films were cut into pieces and analyzed by differential scanning calorimetry. The results are given in Table 2.

TABLE 2

| Sample | T_{g} (° C.) |
|---|---|
| 1 | 25.7 |
| 2 | 10.7 |
| 3 | 3.7 |

### Example 4

### Evaluation of Glycerol Levulinate Ester (GLE) in Paint

In standard formulations shown below in Table 3, GLE was compared with Archer RC. ADM 30-1011 is a low semi-gloss interior white formulation and ADM 40-1022 is a flat interior white formulation.

TABLE 3
Paint Formulation

| | ADM 30-1011 | | | ADM 40-1022 | |
|---|---|---|---|---|---|
| Raw Materials | lbs | gal | Raw Materials | lbs | gal |
| Water | 70.00 | 8.39 | Water | 150.00 | 17.97 |
| Propylene Glycol | 12.00 | 1.39 | Propylene Glycol | 12.00 | 1.39 |
| Tamol 1124 | 5.00 | 0.51 | Tamol 1124 | 9.60 | 0.97 |
| Omyacarb UF | 165.00 | 7.31 | Kathon LX 1.5% | 0.50 | 0.06 |
| Kathon LX 1.5% | 1.75 | 0.21 | Drewplus L = 475 | 4.00 | 0.53 |
| TiO2 Slurry | 260.00 | 13.33 | TiO2 R-902 | 200.00 | 5.99 |
| Water | 60.00 | 7.19 | Snowflake CaCO3 | 230.00 | 10.19 |
| Rhoplex SG-30 | 440.00 | 51.18 | Optiwhite MX | 100.00 | 5.45 |
| Ropaque Ultra Emulsion | — | — | UCAR 379G | 240.00 | 26.52 |
| Coalescent: | 11.27 | 1.48 | Archer RC or GLE | 4.00 | 0.53 |
| Archer RC or (GLE) | | | | | |
| Aerosol OT-75 | 1.50 | 0.16 | Triton GR-5M | 3.60 | 0.42 |
| Byk 1660 | 2.06 | 0.24 | Drewplus L-475 | 4.00 | 0.53 |
| Byk 022 | — | — | Ammonia Water 28% | 4.00 | 0.52 |
| Ammonia Water 28% | 1.50 | 0.20 | Acrysol RM 2020NPR | 12.00 | 1.38 |
| Acrysol 2020NPR | 16.00 | 1.84 | Cellosize HEC ER-52M | 4.00 | 0.96 |
| Acrysol SCT-275 | 6.00 | 0.70 | Water | 222.00 | 26.60 |
| Water | 48.98 | 5.87 | | | |
| Total | 1101.06 | 100.00 | Total | 1199.70 | 100.00 |

The comparative data in Table 4 below show that formulations containing GLE have improved resistance to scrub, block and freeze-thaw. GLE containing formulations also exhibit an increase in gloss. GLE formulations had a lower initial viscosity in semi-gloss (SG) formulation; however, viscosity increased by 26 ku overnight. This increase is approximately 7× that of formulations containing Archer RC. In heat-aged stability testing, the GLE containing paint became cakey after 10 days at 140° F. The formulations containing GLE have lower pH values.

TABLE 4
Paint Properties:

| | | ADM 30-1011 | | ADM 40-1022 | |
|---|---|---|---|---|---|
| Coalescent | | GLE | Archer RC | GLE | Archer RC |
| VOC (g/l) | | 48.29 | 48.11 | 41.32 | 41.26 |
| Lbs/gal | | 11.02 | 11.01 | 12.00 | 12.00 |
| % Solids by weight | | 53.77 | 54.83 | 56.28 | 56.63 |
| % Solids by volume | | 38.77 | 40.26 | 37.03 | 37.56 |
| PVC | | 34.33 | 33.06 | 58.41 | 57.59 |
| Film Finish | | smooth | smooth | smooth | smooth |
| Viscosity, ku/ICI | | 94.1/0.688 | 105.7/0.896 | 92.2/1.108 | 92.0/1.292 |
| Viscosity, ku after | | 119.7 | 109.2 | 96.8 | 95.1 |
| overnight | | | | | |
| pH | | 8.70 | 9.17 | 9.02 | 9.38 |
| Gloss @ | 20 deg | 3.7 | 3.4 | 1.3 | 1.3 |
| | 60 deg | 25.4 | 24.7 | 2.3 | 2.3 |
| | 85 deg | 71.2 | 75.1 | 4.2 | 4.0 |
| Opacity | | 96.55 | 96.57 | 97.0 | 97.13 |
| Color Acceptance | | | | | |
| Draw down ¹ | | | | | |
| Red Oxide dE | | 0.51 | Std | 0.15 | Std |
| Thalo Blue dE | | 0.33 | Std | 0.66 | Std |
| Rub-up ² | | | | | |
| Red Oxide dE | | 0.64 | 1.28 | 0.23 | 0.29 |
| Thalo Blue dE | | 0.46 | 1.23 | 0.09 | 0.33 |
| Heat-aged Stability | | | | | |
| 10 days @ 140° F. | | | | | |
| pH | | Caked | 9.09 | 7.88 | 8.98 |
| Viscosity, ku | | | −1.5 | +3.4 | −0.20 |
| Gloss @ | 20 deg | | 3.6 | 1.3 | 1.3 |
| | 60 deg | | 25.2 | 2.3 | 2.3 |
| | 85 deg | | 76.2 | 4.70 | 3.8 |
| Opacity | | | | | |
| YEᵢₙᵢₜᵢₐₗ | | 3.10 | 2.89 | 3.74 | 3.71 |
| YE_{final} | | — | −2.7 | 3.79 | 3.70 |
| dYE | | — | −5.58 | 0.05 | −0.01 |
| Freeze-thaw ASTM D2243 | | | | | |
| Viscosity, ku | | | | | |
| Cycle 1 | | +18.4 | +17.2 | +5.3 | +8.1 |
| Cycle 2 | | +18.6 | — | +5.9 | +16 |
| Cycle 3 | | +37.0 | — | | |
| Cycle 7 | | — | — | +8.1 | +17 |
| Scrub Cycles | | 2329 | 1725 | 778 | 472 |
| ASTM D2486 | | | | | |
| Block Test ASTM D4946 | | | | | |
| RT | 1 day cure | 10 | 8 | 10 | 10 |
| | 3 day cure | 10 | 9 | 10 | 10 |
| | 7 day cure | 10 | 10 | 10 | 10 |
| 120° F. | 1 day cure | 3 | 3 | 10 | 10 |
| | 3 day cure | 9 | 7 | 10 | 10 |
| | 7 day cure | 9 | 9 | 10 | 10 |

¹ CIELab of Draw downs using Archer RC as Standard
² CIElab of Rub out portions with Drawdown as Standard

### Example 5

### Synthesis of Glycerol Trilevulinate

Amberlyst 35 WET resin was rinsed in anhydrous methanol and dried at 70° C. About 30 g glycerol (0.32 mol, Aldrich), 133.6 g levulinic acid (1.15 mol, Aldrich) were stirred with dried Amberlyst 35 WET resin in a 3-neck round bottom flask. The flask was fitted with a Barrett style receiver, thermocouple and a nitrogen gas inlet. Nitrogen was passed over through the headspace. The mix was heated to 128° C. with moderate stirring until a stoichiometric volume of water was collected (5.8 mL, approximately 2.5 hours). The resulting yellow liquid mixture was allowed to cool to room temperature overnight, then transferred to a separatory funnel. About 50 mL of deionized water was added followed by 200 mL of ethyl acetate. The product was then washed with ten 100 mL aliquots of deionized water, then dried over anhydrous MgSO₄ and contacted with activated carbon to yield a clear liquid. After removal of the ethyl acetate, material having a light amber color and an acid value of 0.6 was obtained.

### Example 6

### Synthesis of Levulinate Esters of Residues of Polysaccharides and Polyols Obtained from Fiber

Corn fiber was obtained from Archer Daniels Midland (Decatur Ill.) and subjected to hydrolysis. Thermochemical hydrolysis of corn fiber (65% moisture) obtained from a corn wet mill (ADM, Decatur, Ill.) was carried out by treating 5 kg with steam in a rotating reactor at 145° C. for 30 minutes. Reaction mixtures were separated with a Rietz horizontal screw press (Minneapolis, Minn.) into a solid fraction (hydrolyzed corn fiber) and a liquid fraction (corn fiber hydrolyzate). The solid fraction (hydrolyzed corn fiber) was then washed with 15 kg of water and the separated with the Rietz horizontal screw press (Minneapolis, Minn.). The wash liquid was pooled with the liquid corn fiber hydrolyzate. Nine batches of 5 kg were treated and pooled to obtain 193 kg of corn fiber hydrolyzate solution. About half of the corn fiber was rendered water soluble by this treatment. Corn fiber hydrolyzate solution (193 kg) was subjected to acid hydrolysis by addition of 1 wt-% sulfuric acid and heating to 121° C. for 30 minutes to yield an acid hydrolyzed corn fiber hydrolyzate. This was concentrated to yield approximately 44.4 kg of 31 wt/wt % acid hydrolyzed corn fiber hydrolyzate concentrate containing 132 grams/liter of organic carbon. Part of the organic carbon (32.5 gram/L) was protein, and a trace (4.4 g/L) of acetic acid was noted. Residues of polysaccharides and polyols in the acid hydrolyzed corn fiber hydrolyzate concentrate which are useful for synthesis of levulinic acid esters are given in Table 6.

TABLE 6
Residues of polysaccharides and polyols
obtained by hydrolysis of corn fiber

| | Concentration | | | Hydroxyls | Moles |
|---|---|---|---|---|---|
| Polyol | (g/L) | MW | moles | per mol | Hydroxyl/Liter |
| Xylose | 53.60 | 144 | .37 | 5 | 1.85 |
| Arabinose | 48.50 | 144 | .34 | 5 | 1.70 |
| Fructose | 0.90 | 180 | 0.005 | 6 | 0.03 |
| Mannose | 1.40 | 180 | 0.008 | 6 | 0.048 |
| Galactose | 8.60 | 180 | 0.048 | 6 | 0.288 |
| Glucose | 57.00 | 180 | .317 | 6 | 1.90 |
| Sucrose | 0.08 | 344 | .0002 | 8 | .0016 |
| Maltose | 2.01 | 344 | .0058 | 8 | 0.046 |
| Total | 175.69 | | | | 5.8636 |
| Polyols | | | | | |

Dried corn fiber hydrolyzate containing residues of polysaccharides and poyols is stirred with a 10% molar excess of levulinic acid (molar excess was calculated in reference to the number of moles of hydroxyl groups in the total polyols) to form a mixture having an acid value A1. About 30 grams dried corn fiber hydrolyzate containing nominally 0.176 moles of hydroxyl groups and sufficient levulinic acid (22.5 grams Aldrich, MW 116.12) to provide a 1.1 molar ratio of levulinic acid to hydroxyl groups are mixed with dried Amberlyst 35 WET resin in a 3-neck round bottom flask This mixture is heated substantially as in Example 1. After the reaction, a product lower in acid value than the value of A1 is obtained. The reaction mixture contains levulinate esters of the residues of polysaccharides and polyols listed in Table 6.

### Example 7

### Synthesis of Levulinate Esters of Residues of Polysaccharides and Polyols Obtained From Fermented Fiber

Acid hydrolyzed corn fiber hydrolyzate concentrate from Example 6 was fermented by two Saccharomyces cerevisiae strains (ADM Y500 and r424a) in two separate continuous fermentations in a set of four New Brunswick BioFlo III fermentors (Edison, N.J.) with a working volume of 2100 mL. The initial fermentation volume in each fermentor was 1500 mL and yeast inoculum was 10%. In one, the fermentation medium was composed of 40% “blender mix” (liquefied starch, backset, corn steep liquor) from the ADM Decatur corn plant (Decatur, Ill.) and 60% acid hydrolyzed corn fiber. All four fermentors were inoculated with 424A (LNHst).

In the second set of four fermentors, the fermentation medium was composed of 80% blender mix and 20% acid hydrolyzed corn fiber hydrolyzate. In the second set, two fermentors were inoculated with ADM y500 and two were inoculated with r424a (LNHst). ADM y500 is a Saccharomyces cerevisiae strain obtained from Archer Daniels Midland (Decatur, Ill.). Saccharomyces cerevisiae r424a (LNHst) was provided by Nancy Ho of the Laboratory of Renewable Resources Engineering at Purdue University, West Lafayette, Ind. One mL of amyloglucosidase (EC 3.2.1.3, ADM, Decatur, Ill.) per liter of fermentation media was added to the fermentors at the start of the fermentation to hydrolyze any maltooligosaccharides remaining in the corn fiber hydrolyzate. The fermentations were run without air addition at 31° C., pH 4.5 (controlled by ammonium hydroxide addition), and agitation was carried out with a single impeller at 150 rpm. The only feeds to the fermentors were blender mix, corn fiber hydrolyzate, and ammonium hydroxide. Samples were taken periodically and the concentrations of polyols were determined by HPLC. The spent fermentation media from both fermentation runs (8 fermentors total) were combined and centrifuged to remove the cell mass and solids. The liquid portion was evaporated in a forced circulation, long-tube vertical evaporator to remove ethanol and some water, to provide a solution containing residues of polysaccharides and polyols (Table 7).

TABLE 7
Residues of polysaccharides and polyols from fermented
acid hydrolyzed corn fiber hydrolyzate

| | Concentration | | | Hydroxyls | Moles |
|---|---|---|---|---|---|
| Polyol | (grams/Liter) | MW | moles | per mol | Hydroxyl/Liter |
| Arabinose | 32.6 | 144 | .226 | 5 | 1.13 |
| Xylose | 28.6 | 144 | .199 | 5 | .995 |
| Sucrose | 0.3 | 344 | .0009 | 8 | .0072 |
| Maltose | 0.2 | 344 | .0006 | 8 | .0048 |
| Isomaltose | 1.9 | 344 | .0055 | 8 | .044 |
| Fructose | 0.8 | 180 | .004 | 6 | .024 |
| Mannose | 0.5 | 180 | .003 | 6 | .018 |
| Galactose | 6.9 | 180 | .038 | 6 | .228 |
| Glucose | 8.9 | 180 | .049 | 6 | .294 |
| Total | 80.7 | | | | 2.745 |

The dried residues of polysaccharides and polyols from fermented acid hydrolyzed corn fiber hydrolyzate are stirred with a 10% molar excess of levulinic acid (molar excess was calculated in reference to the number of moles of hydroxyl groups in the total polyols) to form a mixture having an acid value A1. About 30 grams fermented corn fiber hydrolyzate containing nominally 0.082 moles of hydroxyl groups and sufficient levulinic acid (10.52 grams Aldrich, MW 116.12) to provide a 1.1 molar ratio of levulinic acid to hydroxyl groups are mixed with dried Amberlyst 35 WET resin in a 3-neck round bottom flask. This mixture is treated substantially as in Example 5. After the reaction, a product lower in acid value than the value of A1 is obtained. The reaction mixture contains levulinate esters of the polyols listed Table 7.

### Example 8

### Synthesis of Levulinate Derivatives of Residues of Polysaccharides and Polyols Derived from Hydrogenolysis of Sorbitol

Sorbitol is subjected to hydrogenolysis to provide a mixture of residues of polysaccharides and polyols having 2-5 carbons. The mixture of polyols having 2-5 carbon atoms is analyzed to determine the composition, and the number of hydroxyl groups is determined. A quantity of the mixture of residues of polysaccharides and polyols (X grams) is stirred with sufficient grams of levulinic acid to provide a slight molar excess of levulinic acid with respect to total hydroxyl groups present in the residues of polysaccharides and polyol mixture. The resulting mixture has an acid value A1. This mixture is treated substantially as in Example 6. After the reaction, a product lower in acid value (A2) than the value of A1 is obtained. The reaction mixture contains levulinate esters of residues of polysaccharides and polyols having 2-5 carbon atoms.

Having now fully described this invention, it will be understood to those of ordinary skill in the art that the same can be performed within a wide and equivalent range of conditions, formulations, and other parameters without affecting the scope of the invention or any embodiment thereof.

All documents, e.g., scientific publications, patents, patent applications and patent publications, recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

1. A composition comprising:
a. a polymer; and
b. a coalescent solvent or plasticizer comprising one or more compounds selected from the group consisting of a polysaccharide, a polyol, a residue of a polysaccharide or a polyol, or a mixture thereof directly covalently linked by an O or N with one or more of -(A) or -(G); wherein said -(A) and said -(G) have the following formulae:

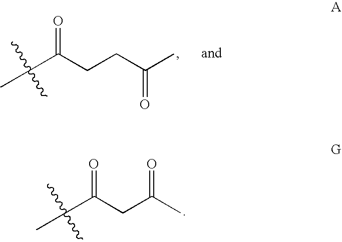

2. The composition of claim 1, wherein said coalescent solvent or plasticizer of (b) comprises one or more compounds having the following structures: 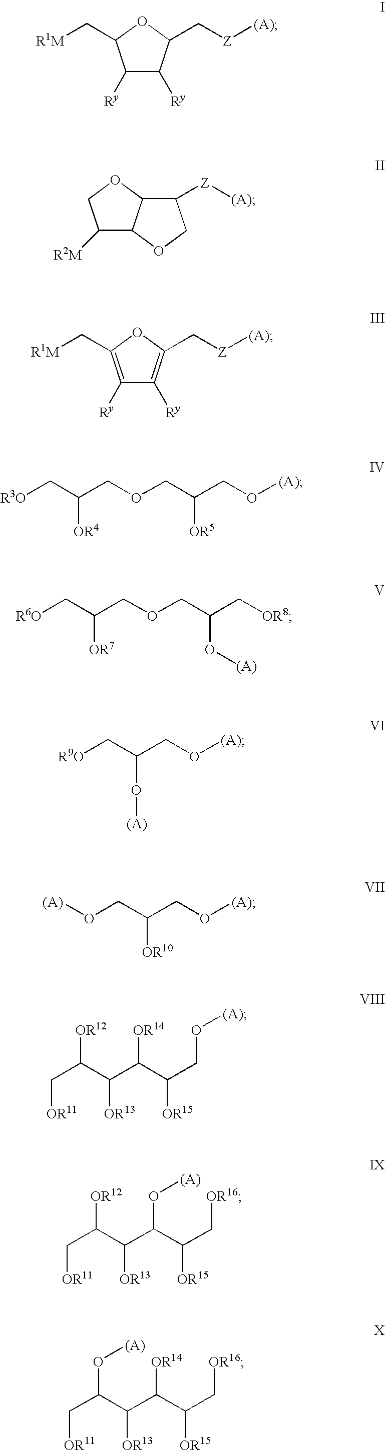
wherein R^{y} in each instance is independently selected from the group consisting of hydroxy, hydrogen, (A)-O—, (G)-O—, C₁₋₄ alkoxy, benzyloxy and benzoyloxy;
wherein R¹, R², R³, R⁴, R⁵, R⁶, R⁷, R⁸, R⁹, R¹⁰, R¹¹, R¹², R¹³, R¹⁴, R¹⁵ and R¹⁶ are each independently selected from the group consisting of hydrogen, (A)-, C₁₋₄ alkyl, benzyl, benzoyl and (G)-;
M=O or N;
Z=O or N,
wherein values of M and Z are independently selected, and M and Z can be the same or different; wherein A, in each instance, is: 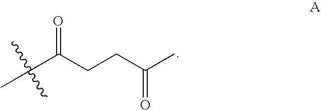
and wherein G, in each instance, is:

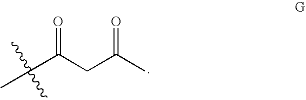

3. The composition of claim 2, wherein said polymer is selected from the group consisting of: polyhydroxyalkanoate, polyvinylchloride, acrylic polymers, styrene-acrylic, vinyl-acrylic, ethylene-vinyl acetate, styrene, polyesters, polyamides, polyethers, polybutadienes, acrylonitrile-butadiene-styrene, styrene-butadiene-styrene, latex polymers and acrylic latex emulsion polymers.

4. The composition of claim 3, wherein said polymer is an acrylic emulsion latex polymer.

5. The composition of claim 4, comprising:
a. an acrylic emulsion latex polymer; and
b. a coalescent solvent or plasticizer comprising one or more compounds having the following structures: 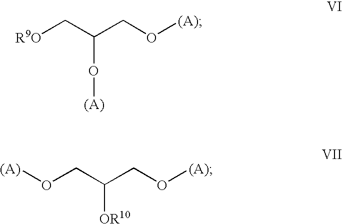
wherein,
R⁹ is selected from the group consisting of hydrogen, (G)- and (A)-; and
R¹⁰ is selected from the group consisting of hydrogen, (G)- and (A)-; wherein (A) and (G) in each instance are as described above.

6. The composition of claim 5, wherein R⁹ and R¹⁰ are each (A)-.

7. The composition of claim 5, wherein said coalescent solvent or plasticizer is present in an amount from about 1% to about 25% w/w of total solids.

8. (canceled)

9. (canceled)

10. The composition of claim 1, wherein the glass transition temperature (T_{g}) of said composition is from about 1° C. to about 40° C.

11. (canceled)

12. (canceled)

13. A composition comprising:
a. a polymer; and
b. a coalescent solvent or plasticizer comprising a compound having the following structure: 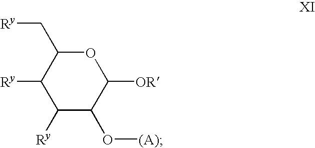
wherein R^{y} is hydroxy;
R′ is; C₁₋₄ alkyl; and
A is:

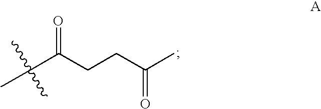

14. (canceled)

15. (canceled)

16. (canceled)

17. (canceled)

18. (canceled)

19. (canceled)

20. (canceled)

21. The composition of claim 13, wherein the T_{g} of said composition is from about 1° C. to about 40° C.

22. (canceled)

23. (canceled)

24. A compound having one of the following formulae: 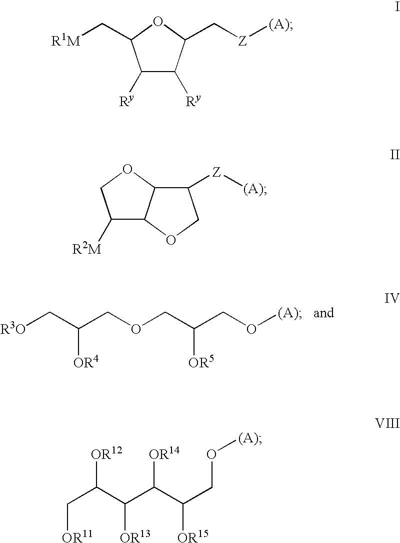
wherein,
R^{y} in each instance is independently selected from the group consisting of hydroxy, hydrogen, (A)-O—, (G)-O—, C₁₋₄ alkoxy, benzyloxy and benzoyloxy;
R¹, R², R³, R⁴, R⁵, R¹¹, R¹², R¹³, R¹⁴, and R¹⁵ are each independently selected from the group consisting of hydrogen, (A)-, (G)-, C₁₋₄ alkyl, benzyl and benzoyl;
M is O;
Z is O;
A, in each instance, is: 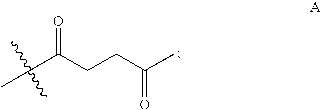
and G, in each instance, is:

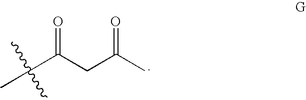

25. (canceled)

26. The compound of claim 24, having Formula I, wherein the compound having Formula I has Formula XX:

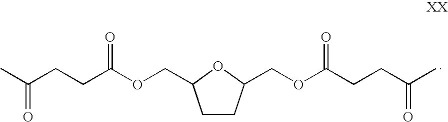

27. The compound of claim 24, having Formula II: 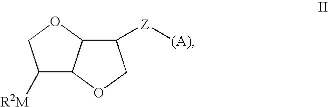
wherein M is O; Z is O; and R² is selected from the group consisting of: hydrogen, C₁₋₄ alkyl, (G)- and (A)-.

28. The compound of claim 27, having Formula XXI:

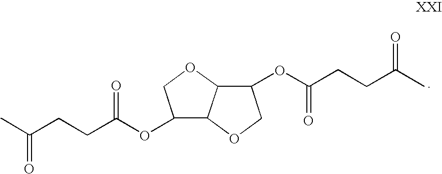

29. The compound of claim 24, having Formula IV: 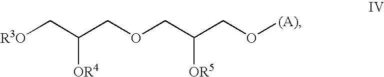
wherein R³, R⁴ and R⁵ are each independently selected from the group consisting of hydrogen, (G)- and (A)-.

30. The compound of claim 29, having Formula XXII:

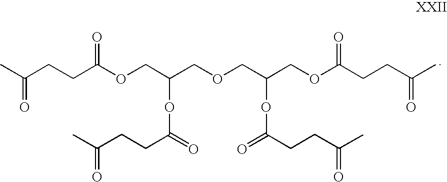

31. The compound of claim 24, having Formula VIII: 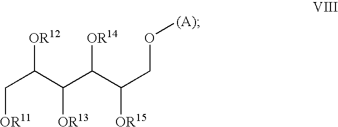
wherein R¹¹, R¹², R¹³, R¹⁴ and R¹⁵ are each independently selected from the group consisting of hydrogen and (A)-.

32. The compound of claim 31, having Formula XXIII: 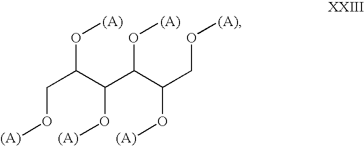
wherein (A), in each instance, is:

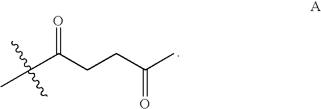

33. A method of making a compound having one of the following formulae: 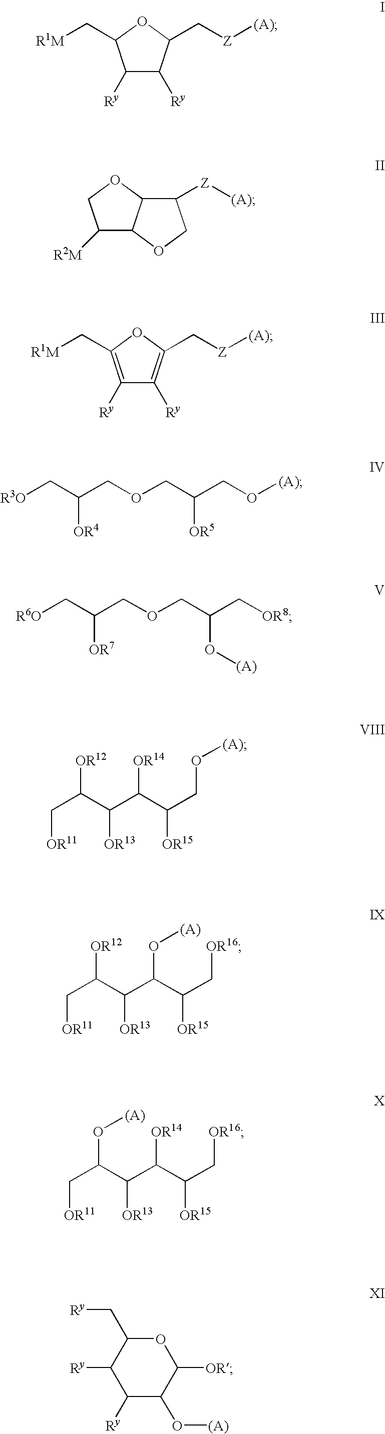 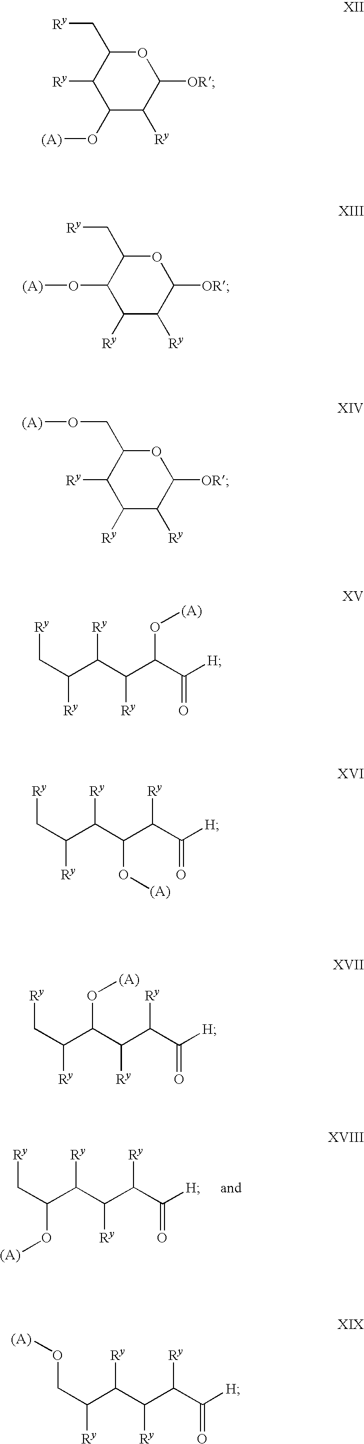
wherein
R^{y} in each instance is independently selected from the group consisting of hydroxy, hydrogen, (A)-O—, (G)-O—, C₁₋₄ alkoxy, benzyloxy and benzoyloxy;
R′ is independently selected from the group consisting of hydrogen, (A)-, (G)-, C₁₋₄ alkyl, benzyl and benzoyl;
R¹, R², R³, R⁴, R⁵, R⁶, R⁷, R⁸, R¹¹, R¹², R¹³, R¹⁵ and R¹⁶ are each independently selected from the group consisting of hydrogen, (A)-, (G)-, C₁₋₄ alkyl, benzyl and benzoyl;
M is O;
Z is O;
A, in each instance, is: 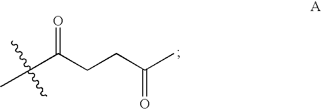
and G, in each instance, is: 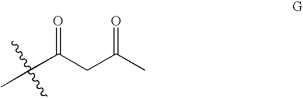
comprising:
a) in the presence of a catalyst, forming a mixture by combining at least one of levulinic acid and levulinic acid ester with one or more of the following compounds: 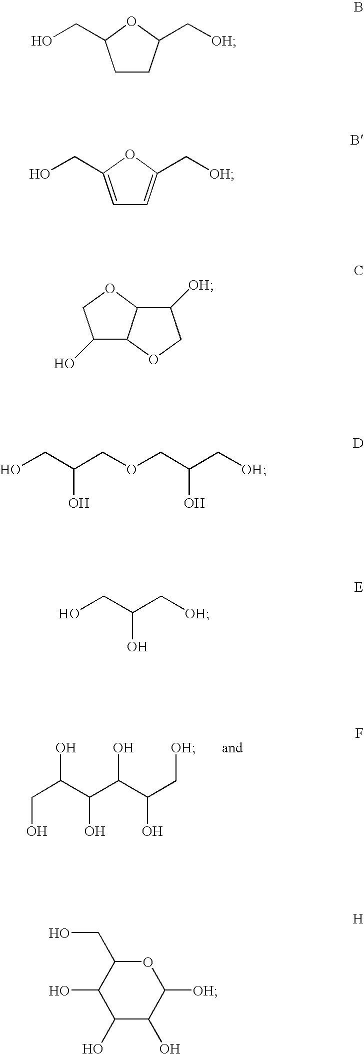
and
b) heating said mixture at a temperature from about 50° C. to about 200° C.,
wherein a compound having one of the above formulae is prepared.

34. A method of making a compound having one of the following formulae: 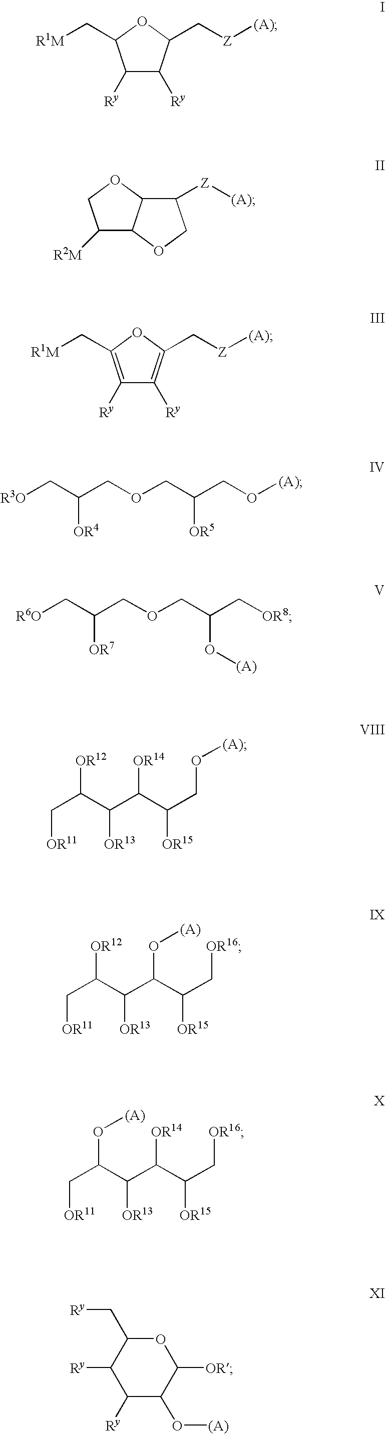 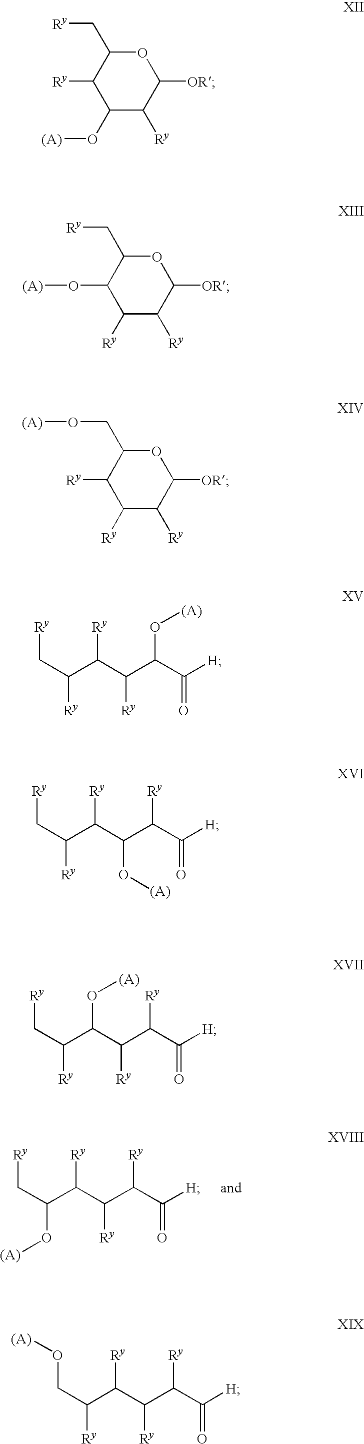
wherein
R^{y} in each instance is independently selected from the group consisting of hydroxy, hydrogen, (A)-O—, (G)-O—, C₁₋₄ alkoxy, benzyloxy and benzoyloxy;
R′ is independently selected from the group consisting of hydrogen, (A)-, (G)-, C₁₋₄ alkyl, benzyl and benzoyl;
R¹, R², R³, R⁴, R⁵, R⁶, R⁷, R⁸, R¹¹, R¹², R¹³, R¹⁴, R¹⁵ and R¹⁶ are each independently selected from the group consisting of hydrogen, (A)-, (G)-, C₁₋₄ alkyl, benzyl and benzoyl;
M is O;
Z is O;
A, in each instance, is: 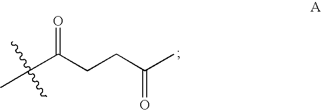
and G, in each instance, is: 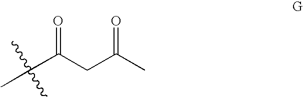
comprising:
a) in the presence of an enzyme catalyst, forming a mixture by combining at least one of levulinic acid and levulinic acid ester with one or more of the following compounds: 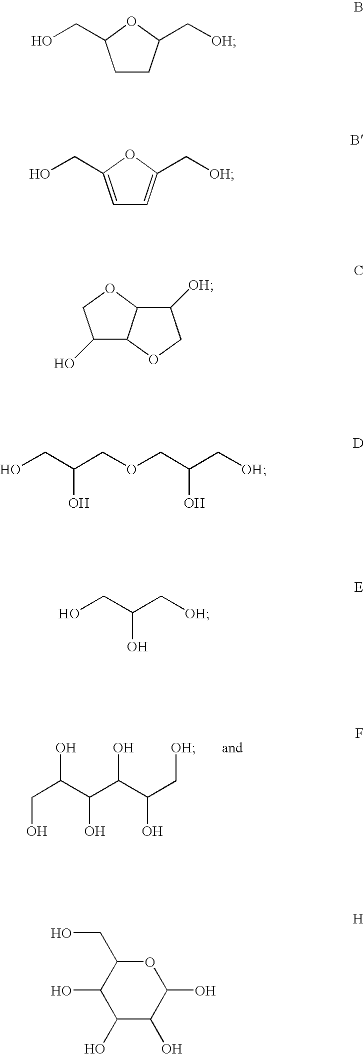
and
b) heating said mixture at a temperature from about 25° C. to about 90° C.,
wherein a compound of claim 24 having one of the above formulae is prepared.

35. (canceled)

36. (canceled)

37. (canceled)

38. (canceled)

39. (canceled)

40. (canceled)

41. The composition of claim 1, wherein said residues of polysaccharides and polyols are prepared by hydrolysis of plant fiber.

42. The composition of claim 41, wherein said plant fiber is corn fiber.

43. The composition of claim 41, wherein said residues of polysaccharides and polyols are selected from the group consisting of arabinose, xylose, sucrose, maltose, isomaltose, fructose, mannose, galactose, and glucose, and combinations and derivatives thereof.

44. The composition of claim 41, wherein said residues of polysaccharides and polyols comprise residues of polysaccharides and polyols prepared by catalytic cracking of mixed polyols.

45. The composition of claim 44, wherein said residues of polysaccharides and polyols comprise residues of polysaccharides and polyols prepared by catalytic cracking of carbohydrates.

46. The composition of claim 41, wherein said residues of polysaccharides and polyols comprise residues of polysaccharides and polyols prepared by catalytic cracking of carbohydrates selected from the group consisting of sorbitol, glucose, sorbitol, mannitol, sucrose, lactose, maltose, alpha-methyl-d-glucoside, pentaacetylglucose and gluconic lactone, and mixtures thereof.

47. The composition of claim 41, wherein said residues of polysaccharides and polyols comprise residues of polysaccharides and polyols prepared by fermenting plant fiber hydrolyzates.

48. The composition of claim 47, wherein said hydrolyzate is corn fiber hydrolyzate.

49. The composition of claim 1, wherein the one or more compounds of (b) further comprises an unsaturated fatty acid directly covalently linked by the carbonyl on said fatty acid to an O or N of said polysaccharide, polyol, said residue of said polysaccharide or polyol, or mixture thereof.

50. The composition of claim 49, wherein said fatty acid is a polyunsaturated fatty acid derived from a vegetable oil.

51. The composition of claim 1, wherein said polysaccharide, polyol, residue of said polysaccharide or polyol, or mixture thereof is starch or a cellulosic polysaccharide or derivative thereof.
